(19) United States
(12) Patent Application Publication
(10) Pub. No.: US 2013/0121459 A1
(43) Pub. Date: May 16, 2013

(54) C-Arm System

(71) Applicant: Michael Meyer, Hausen (DE)

(72) Inventor: Michael Meyer, Hausen (DE)

(21) Appl. No.: 13/672,563

(22) Filed: Nov. 8, 2012

(30) Foreign Application Priority Data
Nov. 10, 2011 (DE) DE 102011086090.8

(51) Int. Cl. A61B 6/00

(52) U.S. Cl. CPC A61B 6/4441
USPC 378/9

(57) ABSTRACT

A C-arm system includes a first floor-mounted C-arm, a second ceiling-mounted C-arm, and a recording space for an object that is to be examined. The C-arms and the recording space are arranged in relation to each other such that recordings of the object may be made by the C-arms in two different planes. The second C-arm is configured for an orbital movement about an angular range of at least 180 degrees.

This application claims the benefit of DE 10 2011 086 090.8, filed on Nov. 10, 2011, which is hereby incorporated by reference.

### BACKGROUND

The present embodiments relate to a C-arm system.

A series of apparatuses exist for imaging processes that play an important role in medical diagnostics, for example. In this situation, the imaging is in many cases carried out by X-rays.

One conventional medical diagnostic appliance based on X-rays is the C-arm. A C-arm may be formed with a C-shaped arm, at the ends of which are arranged an X-ray source and an X-ray detector. As a result of the C-shape, an object that is to be examined may be effectively located between the source and the detector for diagnostic irradiation.

There have been many developments in medical equipment aimed at extending the structure and functions of these devices to accommodate an ever greater range of examination methods.

C-arms have been adapted for orbital rotation about an isocenter (e.g., geometrical mid-point). This orbital movement allows for a plurality of recordings to be taken from different directions of an object positioned in the isocenter. The plurality of recordings or projections from different angles allows for the reconstruction of a three-dimensional image of the object being examined. In order to provide the quality of the 3D reconstruction volume, rotation movements of at least 180° are provided.

Another development has been to combine two or more C-arms. Such systems are described, for example, in U.S. Pat. No. 5,515,416 A1, DE 19533359 C2, and DE 10241184 B4. Most common are biplanar systems, in which two C-arms are used. The C-arms may be mechanically connected or coupled to one another or installed independently of one another. DE 10241184 B4 describes a biplanar system with two separate C-arms that differ in terms of mounting. One of the C-arms is attached using a floor stand, while a ceiling stand is provided for the second C-arm.

Such biplanar systems may be used for cardiology. The parallel use of two C-arms allows for the time duration of a series of recordings to be reduced, and so to better fulfill the requirements incurred by the movement of the heart.

In many cases, C-arm systems are used with several C-arms (e.g., biplanar systems) for a broader spectrum of examinations. Contrary to cardiology applications, with other examinations, only one C-arm may be used.

For example, only one C-arm may be used in angiography in order to carry out vascular examinations. In order to represent blood vessels as 3D structures, fluoroscopic images are produced during the movement of the C-arm. The fluoroscopic images are transferred to an image processing computer. The image processing computer carries out a 3D reconstruction from the plurality of images and may represent the result as a 3D reconstruction volume. The angiography systems available provide for this C-arm movement exclusively in monoplane operation. In other words, if a biplanar system is employed, for example, only one of the C-arms is used. This is illustrated more precisely hereinafter on the basis of FIGS. 2 to 4.

FIG. 2 shows a treatment room with a biplanar system. When the two C-arms 5 and 8 carry out orbital movements, recordings may be taken in two different planes. On the right in FIG. 2, one of the planes has been rotated by 90°, in that the floor-mounted C-arm 8 has been tilted accordingly. Angiography recordings are taken by the first C-arm 8. For this procedure, the second C-arm is moved into the parked position (FIG. 3).

### SUMMARY AND DESCRIPTION

There is a need for C-arm systems that may offer the broadest possible range of examination possibilities.

The present embodiments may obviate one or more of the drawbacks or limitations in the related art. For example, examination possibilities may be extended using conventional C-arm systems.

In one embodiment, a C-arm system for the examination of an object in two planes is provided. The C-arm system includes a first floor-mounted C-arm and a second ceiling-mounted C-arm. In addition, a recording space for an object to be examined is provided. The recording space is realized, for example, by a suitable height-adjustable patient support. The C-arms and the recording space are arranged in relation to one another such that recordings of an object positioned in the recording space may be taken in two planes using the C-arms. In one embodiment, the second ceiling-mounted C-arm is configured so as to carry out an orbital movement about an angular range of at least 180° (e.g., at least 200°).

The C-arm system may be a biplanar system for the examination of an object in two planes. In one embodiment, as well as the two C-arms referred to heretofore, one or more further C-arms are provided, such that recordings may be taken in more than two different planes. The planes may be selected as suitable for the particular application due to the degrees of freedom of the C-arms. The C-arms may not have to be completely rigid C-arms, however. In one embodiment, further degrees of freedom may be provided (e.g., for the movement of arm sections). A C-arm may be characterized by the function of an orbital movement about an isocenter in order to capture recordings in a plane.

In one embodiment, a C-arm system (e.g., a biplanar system), with which rotation movements that are greater than 180° may also be carried out in the second plane, is provided. This allows for the following clinically relevant advantages to be attained.

With the C-arm system, a three-dimensional reconstruction may be carried out in the trunk area of the body. This is not possible with conventional biplanar systems, since the first plane, which may be positioned at the head end, only allows for 3D reconstructions up to a maximum of 900 mm from the head end of the patient. The reason for this is the limited C-arm inner radius of the first C-arm.

During a synchronous C-arm movement of the first plane and the second plane, an angle of at least 180° may be covered by each plane. The images obtained in this way allow for new possibilities in 3D reconstruction to be achieved (e.g., of dynamic processes).

According to a first embodiment, the second C-arm includes an arm that may be moved in a C-shape in the orbital direction, the angular range of which is at least 180° (e.g., at least 200°). The C-shaped segment formed by the arm may have a diameter of, for example <2200 mm, since the distance between the isocenter and the upper edge of the floor does not exceed a distance of 1100 mm (e.g., typical working height for angiography examinations). On account of this diameter, which is smaller than conventional ceiling-mounted C-arms, a mounting part for the second C-arm may be provided. The mounting part may be used to determine the height of the C-arm. The suitable height essentially allows for recordings to be taken about an isocenter in the height range of the recording space. With conventional ceiling heights, the second C-arm may be somewhat lowered. The corresponding distance interval to the ceiling is then realized by the mounting part. The mounting part may also be configured for an adjustment of the height of the C-arm.

According to a second exemplary embodiment, the second C-arm includes a C-shaped arm segment that may be moved in an orbital direction, and a C-shaped arm section arranged on the arm segment. The C-shaped aim section is configured for a movement in the orbital direction relative to the arm segment. With this embodiment, the C-shaped arm section may encompass an angular range of less than 180° (e.g., 120°), but, despite this, by the movement of the movable arm segment, an overall angular range of more than 180° may be attained for recordings of an isocenter. The C-shaped arm section is lengthened by the movable arm segment. During the movement, the angular ranges of the arm segment passed through and the arm section may be added together.

With this exemplary embodiment, the C-shaped arm section may be at least partially moved into the arm segment and accommodated there, which is a space-saving solution. In addition to this, an essentially straight arm section carrying a radiation source or a radiation receiver may be provided to connect to the arm section such that the distance between the corresponding end of the C-arm and the isocenter is reduced. The corresponding arm section with the radiation source or the radiation receiver may also be positioned beneath the object that is to be examined if the radius of the C-shaped arm section is too great for the C-shaped arm section to be positioned beneath the object to be examined and above the floor.

### BRIEF DESCRIPTION OF THE DRAWINGS

FIG. 1 shows a schematic representation of a conventional biplanar system;

FIG. 2 shows a conventional biplanar system in a first position and with a second plane rotated by 90°;

FIG. 3 shows a conventional biplanar system with the second plane in a parked position;

FIG. 4 shows a first embodiment of a C-arm system; and

FIG. 5 shows a second embodiment of a C-arm system.

### DETAILED DESCRIPTION OF THE DRAWINGS

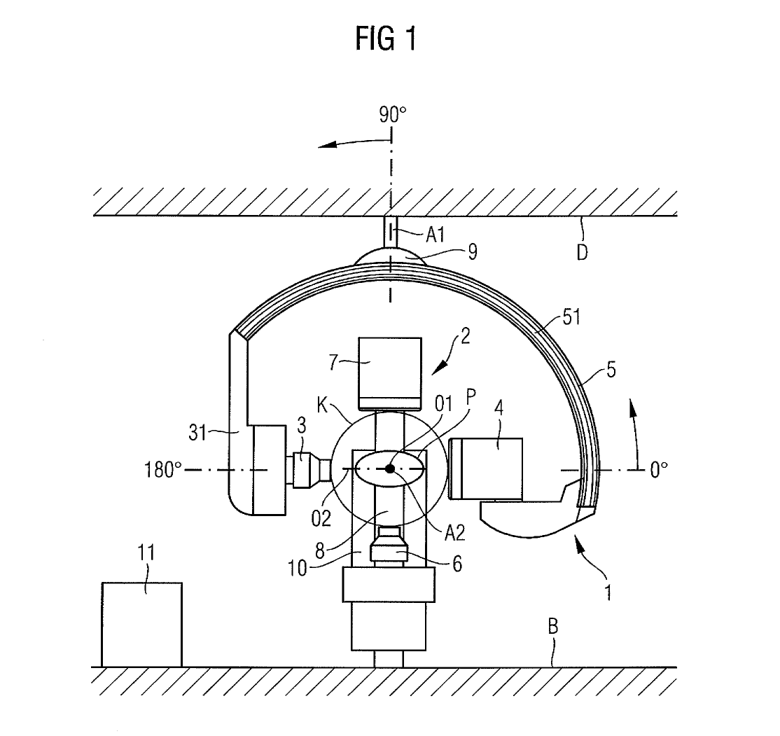

FIG. 1 shows a schematic representation of a biplanar X-ray device. The biplanar X-ray device includes an X-ray system 1 and an X-ray system 2. The X-ray system 1 includes a C-arm 5 having an X-ray radiation source 3 and an X-ray radiation receiver 4. The X-ray system 2 includes a C-arm 8 having an X-ray radiation source 6 and an X-ray radiation receiver 7. The C-arm 5 is arranged on a ceiling D of a space accommodating the biplanar X-ray device, and may be pivoted both about an angulation axis A1 and about an orbital axis O1 at right angles to the plane of the drawing. The pivoting about the orbital axis O1 takes place by moving the C-arm 5 along the circumference of the C-arm 5 in a mounting part 9.

The C-arm 8 of the X-ray system 2 is located or mounted by a mounting (not visible in the figures) on a housing 10 arranged on a floor B of space accommodating the biplanar X-ray device. The C-arm 8 may be pivoted by movement relative to a mounting, about the orbital axis O2 indicated in FIG. 1 and, together with the mounting, about an angulation axis A2 at right angles on the plane of projection.

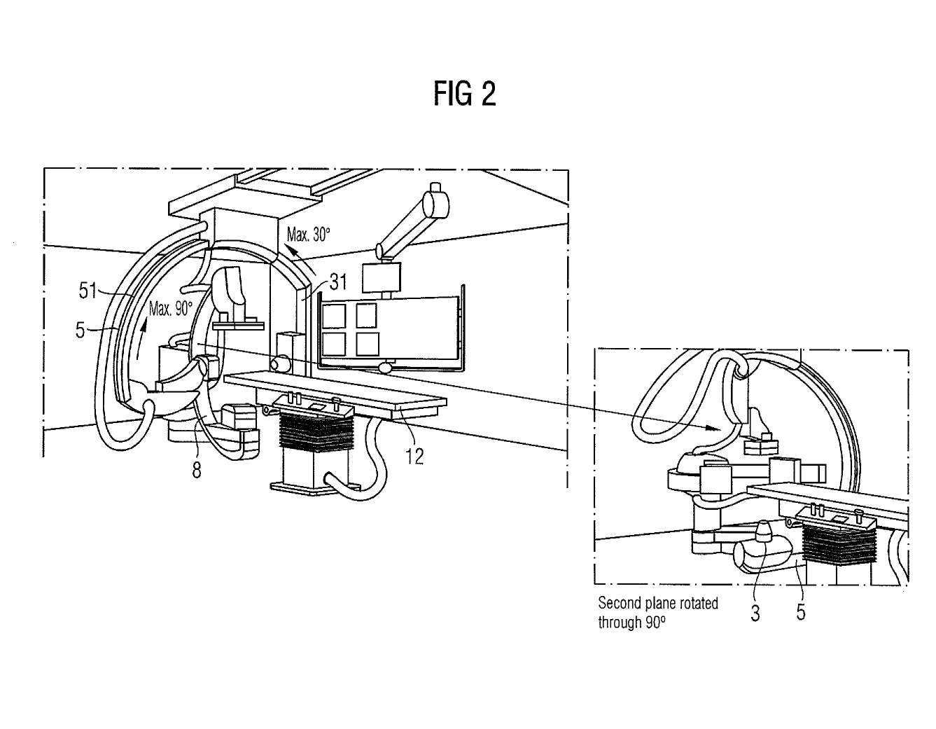

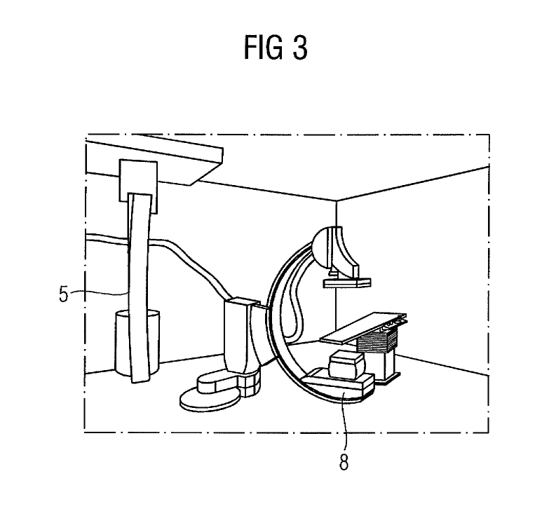

The ceiling-mounted C-arm 5 includes a C-arm-shaped section 51 and an essentially straight section 31 that supports the radiation source 3. Due to the restricted angular range of the C-arm-shaped section 51, the angular range for recordings is restricted by the C-arm 5. This becomes clearer on the basis of FIGS. 2 and 3. Shown in FIG. 2 is a treatment room, in which a conventional biplanar system is arranged. A patient is placed on an accommodation space or a patient couch 12 that allows for recordings by the two C-arms 5 and 8. In the position shown in FIG. 2, due to the restricted length of the C-arm-shaped section 51, the C-arm 5 allows for a maximum rotation about 90° in a clockwise direction and maximum 30° anticlockwise. In other words, the entire angular range is restricted to 120°.

The C-arm 5 includes an angled arm section 31 that allows for the radiation source 3 to be placed below the recording space 12. This is shown on the right in FIG. 2, where the C-arm 5 has been rotated through 90°, such that the essentially straight arm section 31 with the radiation source 3 comes to lie below the recording space 12. FIG. 2 shows that with a lengthening of the C-arm-shaped arm section 51 beyond the 120°, a positioning of the radiation source 3 beneath the recording space 12 may no longer be possible. For angiography examinations, in which, due to the reconstruction quality, images are needed in an angular range of at least 180°, the C-arm 5 secured to the ceiling is therefore positioned in the parked position. The examination is thus carried out by the floor-mounted C-arm 8. Such a scenario is shown in FIG. 3.

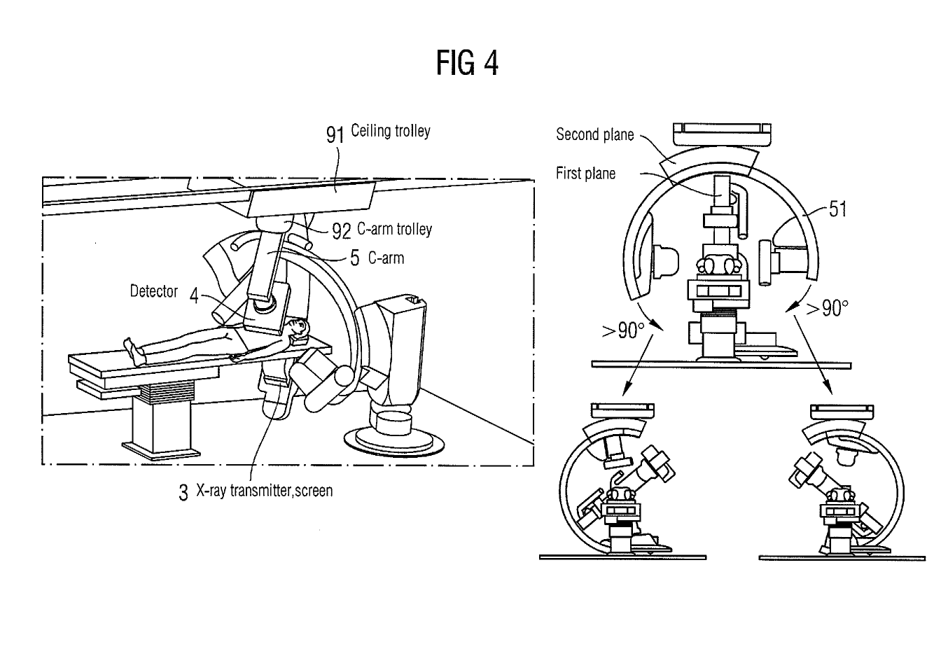

FIG. 4 shows one embodiment of a C-arm system. The C-arm 5 is secured to the ceiling by a ceiling trolley 91 and by a C-arm trolley 92. As is represented on the right side of FIG. 4, an inner radius of the C-arm 5 has been adapted such that the C-arm arm 51 encompasses an angular range of >180°, and a positioning of source and detector, respectively, beneath the radiation source is possible. The outer radius of the C-arm in the second plane is lengthened such that the C-arm may not, under any circumstances, collide with the floor, although a distance between an isocenter and an upper edge of the floor does not exceed a degree of 110 mm (e.g., typical working height for angiography examinations). The C-arm may be mounted lower than is usually the case, which may be achieved by a suitable mounting 9 that is formed by the ceiling trolley 91 and the C-arm trolley 92. If appropriate, such a mounting may also be configured so as to be adjustable in height.

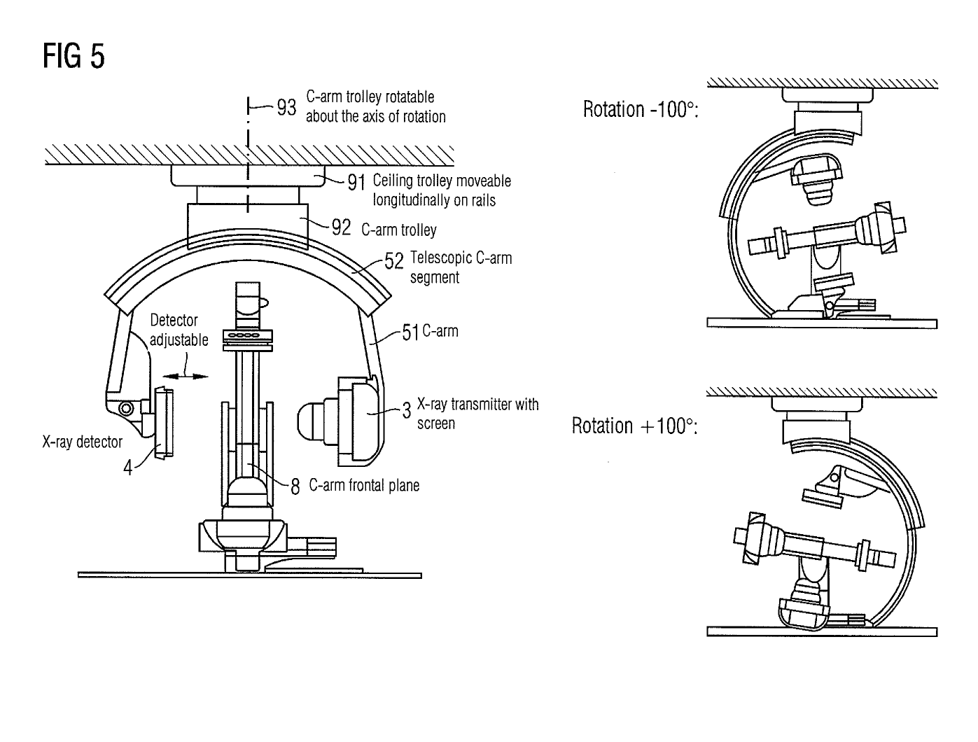

FIG. 5 shows another embodiment of a C-arm system. A C-arm-shaped segment 52 is mounted on the ceiling by a ceiling trolley 91 and a C-arm trolley 92. The C-arm trolley 92 is secured to the ceiling to be rotatable about an axis of rotation 93. The C-arm-shaped arm segment 52 is configured so as to be capable of at least partially accommodating a C-shaped arm section 51. In the left-hand representation in FIG. 5, the C-shaped arm section 51 has almost entirely disappeared in the arm segment 52. A recording angle range of more than 180° is now attained, in that both the arm segment 52 and the arm section 51 arranged on the arm segment 52 move in an orbital direction. In the two representations on the right side of FIG. 6, this is shown for the two extreme settings of minus 100° and plus 100°. In the upper of the two representations, the arm segment 52 has been moved anticlockwise. In addition, likewise anticlockwise, the C-arm-shaped arm section has been moved out, such that, by the compilation or addition of both movements, a recording position of minus 100° is derived. In the lower image, the corresponding action has been carried out for the other direction (e.g., clockwise), such that, for example, a position of 100° is reached. Overall, an angular range of 200° may be passed through with this construction.

The arm segment 52 is configured such that the arm segment 52 may at least partially accommodate the arm section 51 and move the arm section 51 out if required. The functionality is similar to that of a telescope, such that this involves a type of telescopic C-arm segment that is mounted in a telescopic guidance system. Such an arrangement may be provided, but not necessary. Relative mobility in the orbital direction of the arm segment 52 and the arm section 51 is provided. An advantage of this embodiment is that the C-arm shown in FIGS. 1, 2, 3, and 4 may be used. Due to the mounting on an arm segment 52 capable of being moved in an orbital direction, the greater angular range is attained. In other words, the length of the C-shaped arm section 51 of the second plane is shortened such that the arm does not touch the floor even at +/−>90°.

The examples shown are only illustrative of the overall concept. Further embodiments may be evident to the person skilled in the art. For example, the floor-mounted C-arm is not necessarily a rigid C-arm, but may be realized in the form of a two-armed robot with additional degrees of freedom. The exemplary embodiments may therefore not be regarded as being restricted to the scope of protection of the claims.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A C-arm system comprising:
a first C-arm, the first C-arm being floor-mounted;
a second C-arm, the second C-arm being ceiling-mounted; and
a recording space for an object that is to be examined,
wherein the first C-arm, the second C-arm, and the recording space are arranged in relation to each other such that recordings of the object are takeable by the first C-arm and the second C-arm in two different planes, and
wherein the second C-arm is configured for an orbital movement about an angular range of at least 180 degrees.

2. The C-arm system as claimed in claim 1, wherein the second C-arm comprises a C-shaped arm that is moveable in an orbital direction, an angular range of the C-shaped arm being at least 180 degrees.

3. The C-arm system as claimed in claim 2, wherein the angular range of the C-shaped arm is at least 200 degrees.

4. The C-arm system as claimed in claim 2, wherein a C-shaped segment formed by the C-shaped arm has a diameter of less than 2200 mm.

5. The C-arm system as claimed in claim 1, wherein the second C-arm comprises a mounting part, by which a distance is created between the second C-arm and a ceiling that is suitable for examinations about an isocenter essentially in a height range of the recording space.

6. The C-arm system as claimed in claim 5, wherein the mounting part is configured for a height adjustment of the second C-arm.

7. The C-arm system as claimed in claim 3, wherein a C-shaped segment formed by the C-shaped arm has a diameter of less than 2200 mm.

8. The C-arm system as claimed in claim 2, wherein the second C-arm comprises a mounting part, by which a distance is created between the second C-arm and a ceiling that is suitable for examinations about an isocenter essentially in a height range of the recording space.

9. The C-arm system as claimed in claim 3, wherein the second C-arm comprises a mounting part, by which a distance is created between the second C-arm and a ceiling that is suitable for examinations about an isocenter essentially in a height range of the recording space.

10. The C-arm system as claimed in claim 4, wherein the second C-arm comprises a mounting part, by which a distance is created between the second C-arm and a ceiling that is suitable for examinations about an isocenter essentially in a height range of the recording space.

11. A biplanar system comprising:
a C-arm system comprising:
a first C-arm, the first C-arm being floor-mounted;
a second C-arm, the second C-arm being ceiling-mounted; and
a recording space for an object that is to be examined,
wherein the first C-arm, the second C-arm, and the recording space are arranged in relation to each other such that recordings of the object are takeable by the first C-arm and the second C-arm in two different planes,
wherein the second C-arm is configured for an orbital movement about an angular range of at least 180 degrees.
wherein the second C-arm comprises a C-shaped arm segment movable in the orbital direction, and
wherein a C-shaped arm section is arranged on the C-shaped aim segment, the C-shaped arm section being configured for a movement in the orbital direction relative to the C-shaped arm segment.

12. The biplanar system as claimed in claim 11, wherein the C-shaped arm section is at least partially moveable into the C-shaped arm segment.

13. The biplanar system as claimed in claim 11, further comprising an essentially straight arm section carrying a radiation source or a radiation receiver, the essentially straight arm section connecting to the C-shaped arm section, such that a distance between a corresponding end of the second C-arm and an isocenter is reduced.

14. The biplanar system as claimed in claim 12, further comprising an essentially straight arm section carrying a radiation source or a radiation receiver, the essentially straight arm section connecting to the C-shaped arm section, such that a distance between a corresponding end of the second C-arm and an isocenter is reduced.
